(19) United States
(12) Patent Application Publication
(10) Pub. No.: US 2022/0409374 A1
(43) Pub. Date: Dec. 29, 2022

(54) Intracardiac-Echocardiography-based Mitral and Trisucpid Replacement Valve

(71) Applicant: Robert V. Snyders, Ballwin, MO (US)

(72) Inventor: Robert V. Snyders, Ballwin, MO (US)

(21) Appl. No.: 17/850,893

(22) Filed: Jun. 27, 2022

Related U.S. Application Data
(60) Provisional application No. 63/215,443, filed on Jun. 26, 2021.

(51) Int. Cl. A61F 2/24; A61B 8/08; A61B 8/12

(52) U.S. Cl. CPC A61F 2/2469; A61B 8/0883; A61B 8/12; A61F 2230/008; A61F 2240/002

(57) ABSTRACT

A method of constructing a replacement valve for repairing a heart having an annulus separating upstream and downstream regions. The method includes obtaining a representative perimetrical length of the annulus and fabricating a frame having a hub and legs extending outward from the hub to anchors axially offset from the hub. The method includes fabricating an annular band having a circumferential length corresponding to the representative length and attaching the band to the legs. The method includes forming a flexible component having a convex face having a margin and an axially offset region. And a concave face and connecting the offset region to the hub and portions of the margin to the band and/or a portion of the frame. The valve component moves to an open position when upstream pressure is greater than downstream pressure and to a closed position when downstream pressure is greater than upstream pressure.

### CROSS-REFERENCE TO RELATED APPLICATION

Applicant claims the benefit of the co-pending U.S. Provisional Patent Application No. 63/215,443, entitled, “INTRACARDIAC-ECHOCARDIOGRAPHY-BASED MITRAL AND TRICUSPID REPLACEMENT VALVE”, filed on Jun. 26, 2021, which is hereby incorporated by reference in its entirety.

### BACKGROUND

The present disclosure relates generally to heart valve implants, and more particularly, to artificial heart valves custom configured or fitted to repair a particular person's failing heart valve.

A human heart, generally designated in its entirety by the reference character H in FIG. 1, has four chambers that alternately expand and contract to pump blood through vessels extending throughout the body. The chambers consist of a left atrium LA, a left ventricle LV, a right atrium RA, and a right ventricle RV. During each cardiac cycle, muscles of the heart H rhythmically expand and contract the chambers. The heart H includes a check valve at a downstream end of each chamber to ensure blood flows appropriately in a downstream direction through the heart as the heart chambers expand and contract. Oxygen-rich blood enters a left side of the heart H from the lungs (not shown) and leaves through an aorta AA to downstream arteries (not shown) that distribute the blood throughout the body providing oxygen to tissues making up the body. The right atrium RA receives oxygen-depleted blood returning from the body through veins (not shown) and pumps the blood downstream through a pulmonary artery PA to the lungs, which reoxygenate the blood. Because the heart H does not have valves at the upstream ends of the atria, the left atrium LA continuously receives oxygen-rich blood from the lungs, and the right atrium RA continuously receives oxygen-depleted blood returning through the veins.

During a diastole phase of each cardiac cycle, the muscles of the heart H relax, and all four chambers expand. As the left ventricle LV expands, pressure in that chamber drops and a mitral valve MV separating the left atrium LA from the left ventricle opens, allowing blood to flow into the left ventricle from the left atrium LA. Similarly, as the right ventricle RV expands, pressure inside that chamber drops and a tricuspid valve TV separating the right atrium RA from the right ventricle opens, allowing blood to flow into the right ventricle from the right atrium. While both ventricles are relaxed shortly before the end of the diastole phase, the atria contract slightly, pushing additional blood into the ventricles.

During a systole phase of each cardiac cycle, muscles of the heart H contract both ventricles. As the left ventricle LV contracts, pressure in that chamber increases, closing the mitral valve MV and forcing oxygen-rich blood though an aortic valve AV at the downstream end of the left ventricle LV. The blood passing through the aortic valve AV enters the aorta AA and travels downstream through arteries that distribute the oxygen-rich blood throughout the body. Similarly, as the right ventricle RV contracts, the tricuspid valve TV closes and blood is forced through a pulmonary valve PV at the downstream end of the right ventricle into the pulmonary artery PA for transport to the lungs. Tendon-like cords called chordae tendineae CT span the left and right ventricles LV, RV, connecting the mitral valve MV and tricuspid valve TV, respectively, to muscle forming the bottom of the corresponding ventricles. The chordae tendineae CT prevent the leaflets of the mitral valve MV and tricuspid valve TV from opening by prolapsing into the corresponding atrium as the ventricles contract. Accordingly, blood is forced through the correspond downstream valves and does not backflow into the atria. Once the ventricles are fully contracted, the diastole phase rebegins and the downstream valves close.

For a variety of reasons, some mitral valves MV and/or tricuspid valves TV leak, allowing blood to flow back through the valve to the corresponding atrium. When blood flows back into either atrium instead of flowing downstream, insufficient blood is pumped downstream. Various replacement valves have been developed to alleviate leakage. Some replacement valves are bioprosthetic, having animal-based leaflets, e.g., harvested bovine or porcine heart valves, mounted in a frame adapted to be implanted in the heart to replace the leaking valve. Wholly artificial valves, i.e., mechanical valves, have also been developed to replace failing valves. Like the animal-based replacement valves, most artificial valves have leaflets mimicking native valves mounted in a frame that is implanted in the heart.

Although native mitral valves MV and tricuspid valves TV are irregularly shaped (i.e., non-circular) as shown in FIG. 2, current replacement valve frames are typically circular. When the frames of these replacement valves are distorted, the leaflets become misaligned when the valve closes so their corresponding sealing surfaces do not meet precisely. This misalignment causes the replacement valves to leak. To alleviate this problem, surgeons make sutures or install clips (not shown) in tissue surrounding a replacement valve, so the altered tissue forms a generally circular opening that conforms to the undistorted circular shape of the replacement valve frame. Making these alterations, however, is time consuming and difficult to accomplish in beating heart procedures. Further, these surgical alterations are subject to failure, allowing blood to pass between the frame and tissue and distorting the frame so the replacement valve leaks. Even when the alterations do not fail, they reduce flow area thereby restricting blood flow through the heart. Accordingly, there is a need for a method of constructing a replacement valve that does not leak when distorted so the replacement valve can conform to a patient's heart anatomy without adverse effects.

Although some replacement valves are less susceptible to leakage when distorted, surgeons often must alter the surrounding tissue to match the configuration of the replacement valve to prevent leakage between the tissue and the replacement valve. These alterations introduce many of the same problems discussed above. Thus, there is a need for a replacement valve that is sized to correspond to the particular patient's heart, so surgeons need not alter surrounding heart tissue and restrict flow area.

As many as half of mitral valve replacement currently are performed using conventional surgery during which a patient's chest be opened and heart is bypassed while implanting the valve. These procedures are invasive and surgically traumatic, increasing recovery time and potential for fatality, particularly with older patients, which are most likely to need mitral valve replacement. Another procedure currently being investigated involves making smaller incisions into the chest cavity and through heart muscle into a patient's left ventricle to insert a catheter that delivers a replacement valve (e.g., a Tendyne mitral replacement valve available from Abbott Laboratories Medical Device Company). The valve opens inside the left atrium before being tethered to the heart muscle where the valve entered the left ventricle. This procedure potentially increases left ventricular loading, obstructs left ventricular outflow, causes blood loss, and other problems. Although the transcatheter replacement valve may be less invasive and reduce surgical trauma compared to conventional surgery, the valve does not minimize these problems and potentially introduces others.

### SUMMARY

In one aspect, the present disclosure includes a method of constructing a replacement valve for repairing a patient's failing heart valve. The failing valve has an annulus separating an upstream region from a downstream region. The method includes obtaining a representative inner perimetrical length of the annulus. A frame having a central hub and a plurality of flexibly resilient legs extending radially outward from the central hub is fabricated. Each leg extends to an anchor axially offset from the central hub by a preselected distance. Another step of the method includes fashioning an annular band having an outer circumferential length corresponding to the representative inner perimetrical length of the annulus. The band is attached to at least a portion of the plurality of legs of the frame adjacent to the respective anchors to limit spacing between the respective anchors of adjacent legs. A flexible valve component having a convex face and a concave face opposite the convex face is formed. The convex face has an annular margin and a central region axially offset from the annular margin. The central region of the convex face is connected to the central hub of the frame and circumferentially spaced portions of the annular margin are connected to at least one of the band and a portion of the frame adjacent to the anchors. The valve component is adapted to move when repairing the patient's failing heart valve in response to a difference between fluid pressure in the upstream region and fluid pressure in the downstream region. The valve component moves relative to the band between an open position in which the valve component permits downstream flow between the band and the annular margin of the valve component and a closed position in which the valve component blocks upstream flow between the band and the annular margin of the valve component. The valve component moves to the open position when fluid pressure in the upstream region is greater than fluid pressure in the downstream region to permit downstream flow from the upstream region to the downstream region. And the valve component moves to the closed position when fluid pressure in the downstream region is greater than fluid pressure in the upstream region to prevent flow reversal from the downstream region to the upstream region.

In another aspect, the present disclosure involves a method of performing a medical activity to repair a patient's failing heart having an annulus separating an upstream region from a downstream region. The method comprises the step of performing intracardiac echocardiography to measure a representative inner perimetrical length of the annulus. Further, the method includes the step of constructing a replacement valve. The replacement valve is constructed by fabricating a frame having a central hub and a plurality of flexibly resilient legs extending radially outward from the central hub. Each leg of the plurality of legs extends to an anchor axially offset from by a preselected distance the central hub. Another step of the method includes fashioning an annular band having a circumferential length corresponding to the representative inner perimetrical length of the annulus. The method also includes attaching the band to at least a portion of the plurality of legs of the frame adjacent to the respective anchors to limit spacing between the respective anchors of adjacent legs of the plurality of legs. A flexible valve component having a convex face and a concave face opposite the convex face is formed. The convex face has an annular edge margin and a central region axially offset from the edge margin. Moreover, the central region of the convex face is connected to the central hub of the frame and portions of the edge margin to at least one of the band and a portion of the frame adjacent to the anchors. The valve component is substantially free of connections to the frame other than the central hub and adjacent to the anchors. The valve component is adapted to move when repairing the patient's failing heart valve in response to a difference between fluid pressure in the upstream region and fluid pressure in the downstream region. The valve components move relative to the band between an open position in which the valve component permits downstream flow between the band and the edge margin of the valve component and a closed position in which the valve component blocks upstream flow between the band and the edge margin of the valve component. The valve component moves to the open position when fluid pressure in the upstream region is greater than fluid pressure in the downstream region to permit downstream flow from the upstream region to the downstream region. The valve component moves to the closed position when fluid pressure in the downstream region is greater than fluid pressure in the upstream region to prevent flow reversal from the downstream region to the upstream region. The method further comprises performing heart surgery to implant the constructed replacement valve in the patient's failing heart with the annular band of the replacement valve aligned with the measured annulus separating the upstream region from the downstream region and the flexible valve component oriented so the valve component moves to the open position when fluid pressure in the upstream region is greater than fluid pressure in the downstream region and the valve component moves to the closed position when fluid pressure in the downstream region is greater than fluid pressure in the upstream region.

In yet another aspect, the present disclosure includes a method of constructing a replacement valve for repairing a patient's failing heart valve having an annulus separating an upstream region from a downstream region that has a known representative inner perimetrical length. The replacement valve is constructed by fabricating a frame having a central hub and a plurality of flexibly resilient legs extending radially outward from the central hub. Each leg of the plurality of legs extends to an anchor axially offset from by a preselected distance the central hub. An annular band having a circumferential length corresponding to the representative inner perimetrical length of the annulus is fashioned. The method also includes attaching the band to at least a portion of the plurality of legs of the frame adjacent to the respective anchors to limit spacing between the respective anchors of adjacent legs of the plurality of legs. A flexible valve component having a convex face and a concave face opposite the convex face is formed. The convex face has an annular edge margin and a central region axially offset from the edge margin. Moreover, the central region of the convex face is connected to the central hub of the frame and portions of the edge margin to at least one of the band and a portion of the frame adjacent to the anchors. The valve component is substantially free of connections to the frame other than the central hub and adjacent to the anchors. The valve component is adapted to move when repairing the patient's failing heart valve in response to a difference between fluid pressure in the upstream region and fluid pressure in the downstream region. The valve components move relative to the band between an open position in which the valve component permits downstream flow between the band and the edge margin of the valve component and a closed position in which the valve component blocks upstream flow between the band and the edge margin of the valve component. The valve component moves to the open position when fluid pressure in the upstream region is greater than fluid pressure in the downstream region to permit downstream flow from the upstream region to the downstream region. The valve component moves to the closed position when fluid pressure in the downstream region is greater than fluid pressure in the upstream region to prevent flow reversal from the downstream region to the upstream region.

Other aspects of the disclosure will be apparent in view of the following description, including claims and drawings.

### BRIEF DESCRIPTION OF DRAWINGS

FIG. 1 is a front elevational cross section of a human heart during a diastole phase of a cardiac cycle when heart muscle relaxes and valves separating ventricles from atria are open;

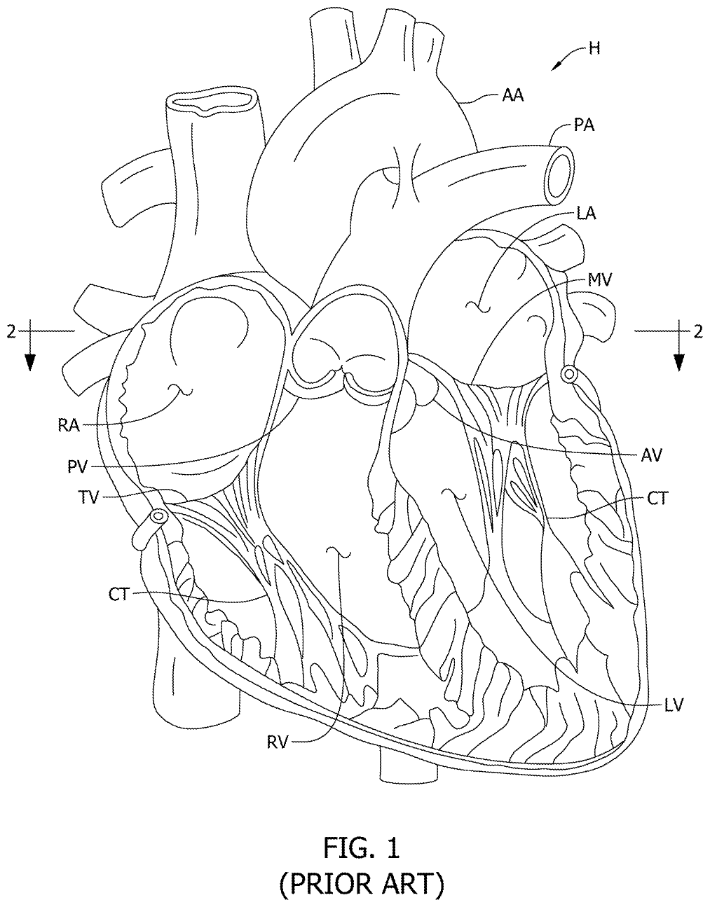

FIG. 2 is a section of the hear taken in the plane of line 2-2 of FIG. 1 when transitioning from a systole phase to a diastole phase during which all heart valves are closed;

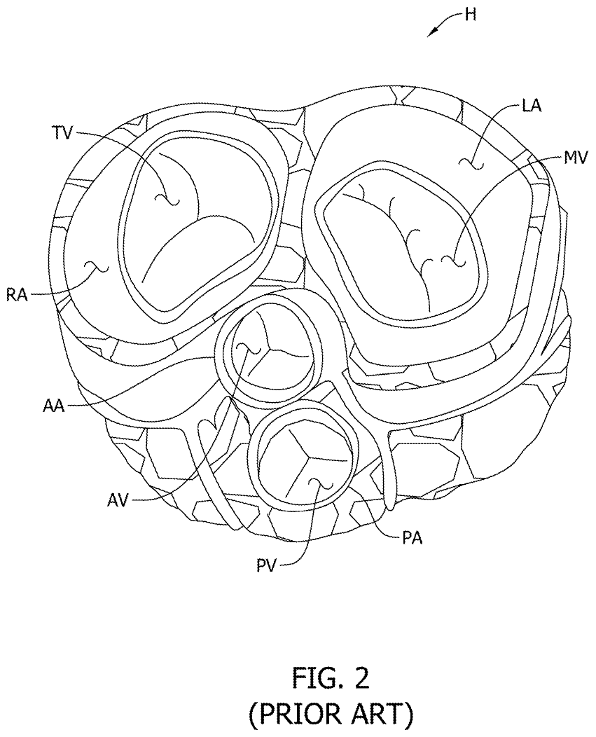

FIG. 3 is a partially sectional elevation of a replacement valve;

FIG. 4 is a top plan of the valve of FIG. 3;

FIG. 5A is a fragmentary bottom plan of the valve of FIG. 3 in a closed configuration;

FIG. 5B is a bottom plan of the valve in an open position;

FIG. 6 is a front elevational cross section of a heart showing a fitted replacement valve in position to replace a native tricuspid valve;

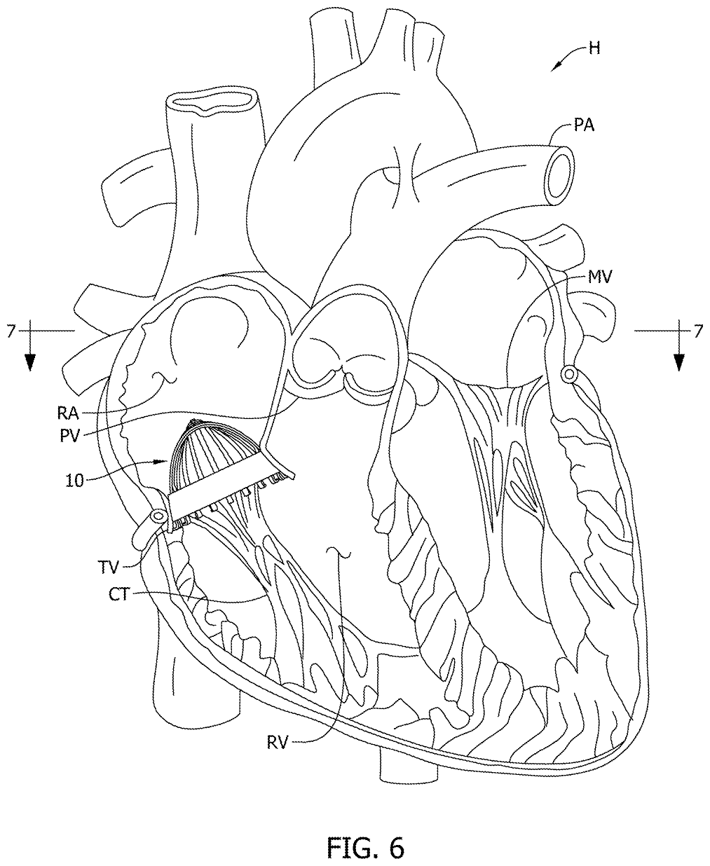

FIG. 7 is a section of the heart and valve taken in the plane of line 7-7 of FIG. 6; and

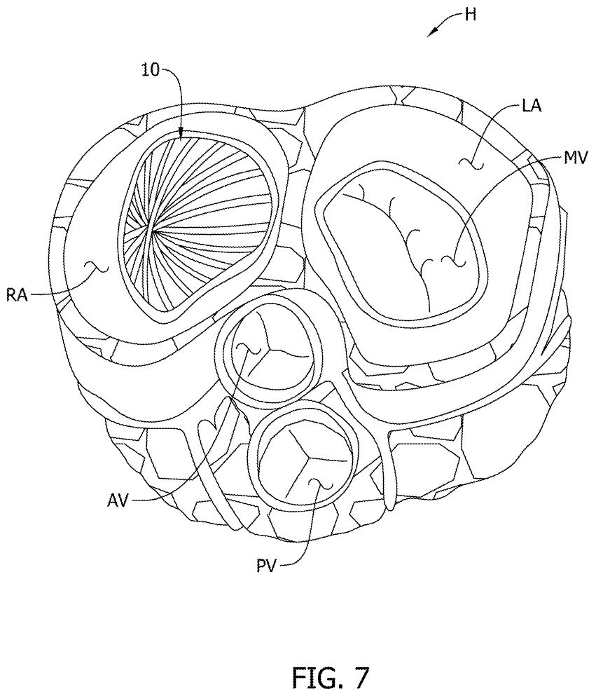

FIG. 8 is a front elevational cross section of a heart showing a fitted replacement valve in position to replace a native mitral valve.

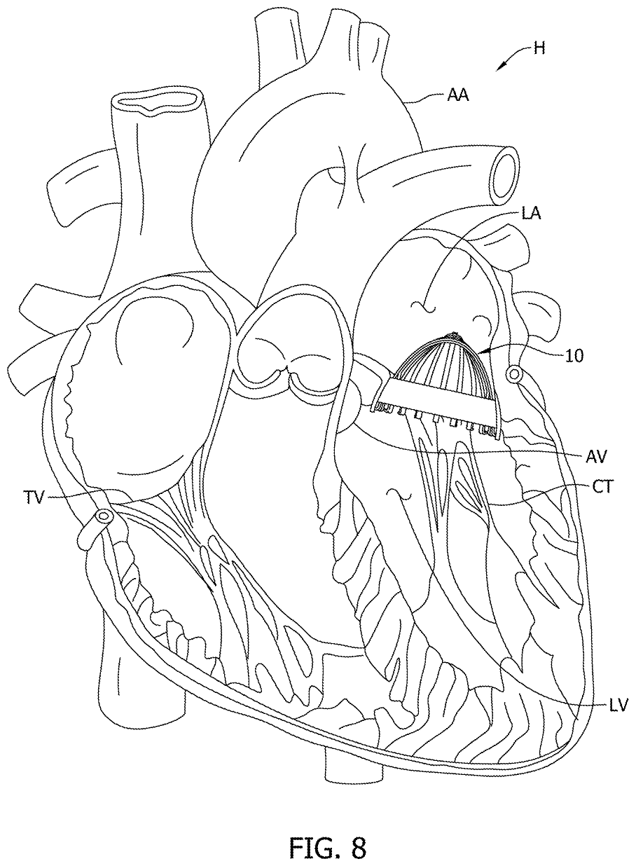

Corresponding reference characters indicate corresponding parts throughout the drawings.

### DETAILED DESCRIPTION

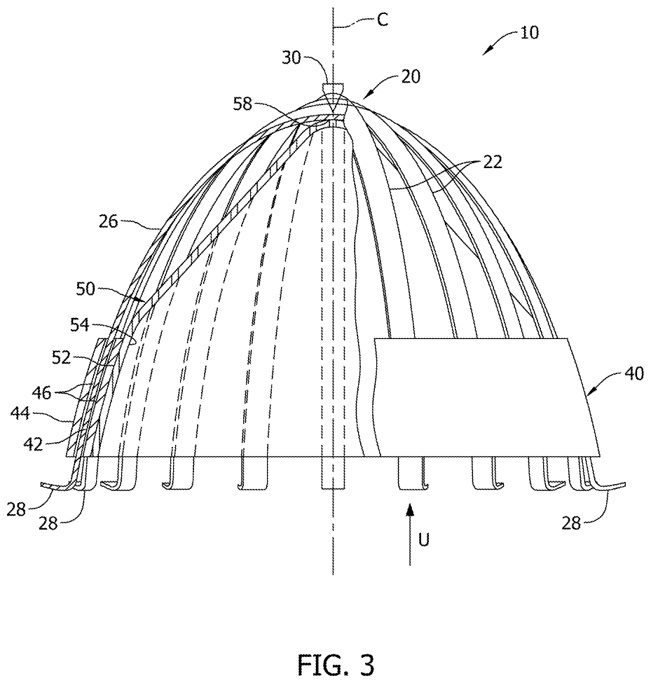

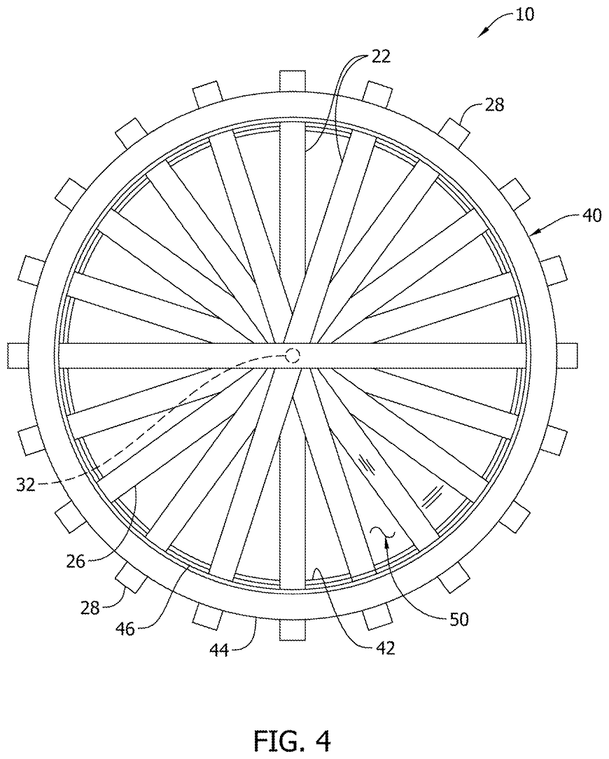

As illustrated in FIGS. 3 and 4, an artificial heart valve is designated in its entirety by the reference number 10. The valve 10 comprises a flexibly resilient external frame, generally designated by 20. The frame 20 is fabricated by joining a plurality of U-shaped elements 22. As shown in FIG. 4, each U-shaped element 22 has a center portion, generally designated by 24, positioned midway along the element and two flexibly resilient legs 26 extending radially outward in opposite directions from the center portion. Each leg 26 also extends longitudinally downstream to a corresponding foot or anchor 28 that is configured for engaging tissue in the heart when the valve 10 is implanted as will be explained. Although the U-shaped elements 22 may be made of other materials, each of the illustrated elements comprises a nitinol superelastic alloy wire. In the illustrated example, the wire has a rectangular cross section with a width of about 3 mm and a thickness of about 0.1 mm to about 0.2 mm. Although the elements 22 may be joined using other methods such as with adhesive, in the illustrated example the elements are joined midway along their lengths by brazing to create a central hub 30. In some examples, it is envisioned that the central hub 30 may include a connector 32 (indicated with dashed lines in FIG. 4) for connecting the valve 10 to instruments when positioning the valve in a patient's heart. Although the connector 32 may have other configurations, an exemplary connector comprises an externally threaded micro-screw post. In some examples (not shown), the connector 32 includes a central opening to allow a small amount of fluid to pass upstream when the valve is closed. As those skilled in the art will appreciate, it is envisioned that the central opening may prevent fluid from stagnating adjacent to a downstream face of the valve. In the illustrated example, each anchor 28 is axially offset from the central hub by a preselected uniform distance. In addition, the frame 20 illustrated in FIGS. 3 and 4 comprises ten elements 22 providing twenty legs 26 oriented so the feet 28 are generally equally spaced about an imaginary longitudinal centerline C extending through the central hub 30. It is envisioned that the frame 20 may have other numbers of elements, legs, and anchors. It is also envisioned that the frame 20 may have a unitary construction.

It is envisioned that each anchor 28 may taper from a width of about 3 mm to a width of about 2 mm or less, allowing the anchor to penetrate tissue in the heart more easily when being implanted. Further, each anchor 28 is angled perpendicular to the centerline C or angled upstream (i.e., in a direction corresponding to the arrow U in FIG. 3). In some examples, the anchors may be angled upstream by about 30° or more. Thus, the anchors 28 may make an angle in a range from about 60° to about 90° relative to the centerline C to prevent the valve 10 from migrating when pressure upstream from the valve 10 falls below pressure downstream. In most cases, the anchors 28 do not have barbs, permitting the valve 10 to be collapsed and repositioned during implantation.

As further illustrated in FIGS. 3 and 4, an annular band, generally designated by 40, extends around the legs 26 of the frame 20 adjacent to the anchors 28. The band 40 limits maximum spacing between adjacent legs 26 and anchors 28. In the illustrated example, the elements 22 are annealed so they are biased toward straightening. The band 40 prevents the legs 26 from straightening thereby inducing a bending load in the elements 22 that increases the buckling strength under longitudinal loading of the dome-shaped frame configuration. The band 40 is sufficiently flexible to permit the frame elements 22 to be gathered together so the legs and anchors are closer to the centerline C for implanting the valve 10 transvenously.

Although the band 40 may have other constructions, the illustrated band has an annular inner strip 42 positioned inside the legs 26 and an annular outer strip 44 surrounding the legs so the inner strip and outer strip are joined in face-to-face relation and the legs are sandwiched between the inner and outer strips as illustrated in FIGS. 3, 4, 5A, and 5B. Although the inner strip 42 and outer strip 44 may be joined to each other and attached to the frame 20 using other means, the strips of the illustrated example are adhesively bonded to each other and to the frame by an adhesive layer 46. Further, although the band 40 illustrated in FIG. 3 is substantially cylindrical or slightly tapered, it is envisioned the band may have other shapes. For example, it is envisioned the band 40 may include a rim or a flange (not shown) extending circumferentially around a downstream edge to sealingly engage tissue of the heart.

The illustrated inner strip 42 comprises a non-porous woven Dacron® polyester to provide a smooth surface for reducing surface friction and to enhance sealing properties, and the illustrated outer strip 44 is a porous knit Dacron® polyester to promote vascularization and tissue ingrowth into the band for enhancing stability of the valve 10 after being implanted. (Dacron is a U.S. federally registered trademark owned by Invista North America, LLC of Wichita, Kans.) It is also envisioned that the band 40 may comprises other suitable materials such as heterologous animal pericardium (e.g., bovine or porcine pericardium), autologous tissue engineered substrates, or a biocompatible, radiopaque, elastic material, such as silicone rubber, polyurethane, or polytetrafluoroethylene (PTFE).

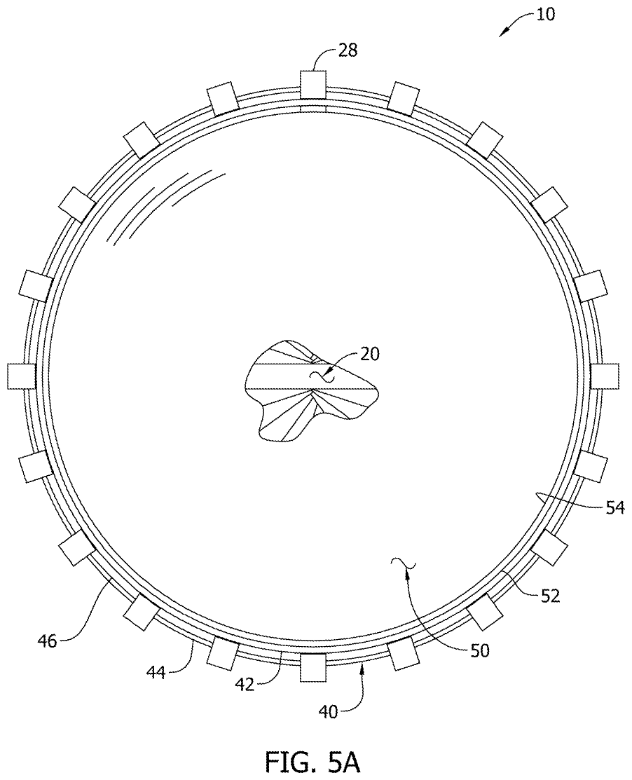

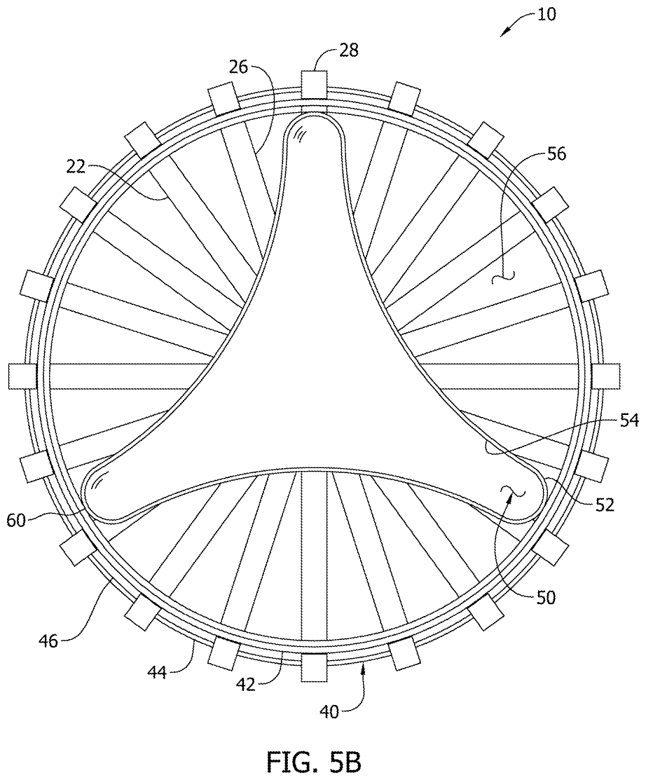

As shown in FIGS. 5A and 5B, a flexible valve component, generally designated by 50, is disposed within the frame 20 and attached to a downstream face of the central hub 30. Although the component 50 may have other shapes, the illustrated component is generally conical when not deformed, resulting in the component having a generally convex face 52 and a generally concave face 54. When implanted, the convex face 52 faces upstream and the concave face 54 faces downstream. When the frame 20 is anchored within the annulus of a failing heart valve in a position between an upstream region and a downstream region, the valve component 50 moves in response to differences between fluid pressure in the upstream region and the downstream region between a closed position (as shown in FIG. 5A) and an open position (as shown in FIG. 5B). When the valve component 50 is in the open position, it moves inward toward the centerline C of the valve 10, creating openings, generally designated by 56, between the valve component and the inner strip 42 to allow blood flow between the upstream region and the downstream region. When in the closed position, the valve component 50 expands away from the centerline C and sealingly engages the inner strip 42 to block flow between the upstream and downstream regions. The valve component 50 moves to the open position when fluid pressure in the upstream region is greater than fluid pressure in the downstream region to permit downstream flow from the upstream region to the downstream region. The valve component 50 moves to the closed position when fluid pressure in the downstream region is greater than fluid pressure in the upstream region to prevent flow reversal from the downstream region to the upstream region. Although the valve component 50 may be made of other materials, the illustrated valve component is made of a biocompatible elastic material such as silicone rubber, polyurethane, PTFE, heterologous animal pericardium (e.g., bovine or porcine pericardium), or autologous tissue engineered substrates.

The upstream side 52 of the flexible valve component 50 has an apex 58 that is attached to the downstream face of the frame 20 at the central hub 30. Although the valve component may be attached to the frame 20 by other means, the illustrated valve component 50 is attached to the frame by adhesive bonding. Further, the flexible valve component 50 is attached to the frame 20, and more specifically to the band 40, at several attachment points 60 around the frame. In the illustrated example, the valve component 50 is attached to the band 40 at three generally equally spaced locations around the band. Thus, the valve component 50 forms flaps 62 extending between adjacent attachment points 60. Each of the flaps 62 and a corresponding portion of the band 40 extending between adjacent attachment points 58 define one of the openings 56 through the valve when the valve component 50 moves to the open position, with the flaps of the valve component pushed inward toward the centerline C. Although the valve component may be attached to the band 40 by other means such as thermal bonding, ultrasonic bonding, laser enhanced bonding, and sewing, the illustrated valve component 50 is attached to the band by adhesive bonding.

Before making a replacement valve 10 for a particular patient's failing heart valve, a perimetrical length of a portion of the patient's heart must be measured. It is envisioned that the failing valve may be a native heart valve or a previously implanted replacement valve. Although the failing valve may correspond to a pulmonary valve PV or an aortic valve AV, in most instances it is envisioned the valve will be a mitral valve MV or a tricuspid valve TV. Accordingly, the required measurement will be described with respect the mitral valve and the tricuspid valve. Each of these valves separates an upstream region from a downstream region. For example, a mitral valve MV separates a left atrium LA (broadly, an upstream region) from a left ventricle LV (broadly, a downstream region). The mitral valve MV extends inward from tissue separating the upstream region from the downstream region. In cases where the replacement valve will be replacing a native mitral valve MV, a surgeon may prefer to leave the failing mitral valve leaflets intact. The required measurement will be an inner perimetrical length of an annulus including the tissue surrounding the mitral valve and the leaflets of the mitral valve MV fully opened against the tissue. In cases where the surgeon opts to remove the leaflets before implanting the replacement valve, the required measurement will be an inner perimetrical length of an annulus of heart tissue remaining after the leaflets are removed. In cases where the valve will be replacing a previously implanted replacement valve, the required measurement will be an inner perimetrical length of the frame of the previously implanted replacement valve with the valve elements (e.g., leaves) fully opened or removed as the surgeon prefers. From these examples, it is believed one skilled in the art will be able to determine which annulus should be measured to determine a corresponding inner perimetrical length. It is envisioned that any suitable procedure may be used to estimate the perimetrical length of the appropriate annulus. In one example, the patient's heart is imaged using intracardiac echocardiography (ICE). A two-dimensional planimetric analysis is performed using the resulting image to determine the nominal perimetrical length for the particular heart valve being replaced (e.g., the mitral valve MV or tricuspid valve TV). One benefit of using the planimetric analysis is that the two-dimensional analysis disregards three-dimensional variations that may be present in the ICE image so the fabricated heart valve 10 is appropriately sized to avoid leaks around the valve when implanted.

Once the perimetrical length of the annulus is determined, a skilled technician will be capable of making the replacement valve 10 for the particular failing heart valve. The frame 20 is fabricated using conventional methods such as those described above. Each frame element 22 is selected such that its size and shape will not interfere with operation of the upstream chamber. Once the frame 20 is fabricated, the annular band 40 may be fashioned by forming an annular outer strip 44 having an outer circumferential length equal to the representative inner perimetrical length of the failing valve annulus previously measured. The outer strip 44 is positioned around the legs 26 of the frame 20 adjacent to the anchors 28. The inner strip 42 is formed so its outer circumferential length corresponds to the inside surface of the outer strip 44 and the legs 26. The inner and outer strips 42, 44, respectively, are joined in face-to-face relation and to the frame 20 as described previously. The flexible valve component 50 is formed as previously discussed so that the component has an upstream side 42 and a downstream side 44. The apex 58 of the upstream side 42 is attached to the downstream face of the frame 20 at the central hub 30, and downstream points 60 on the convex side 42 of the component 50 are attached to corresponding points on the inner surface of the inner strip 42. Once the technician makes the valve 10, which is custom sized for the particular failing heart valve, the replacement valve may be implanted in the heart H using a suitable procedure. Such procedures are within the ordinary skill of practitioners in the art.

When introducing elements in the present written disclosure and appended claims, the articles “a”, “an”, “the”, and “said” should be interpreted as meaning there are one or more of the elements. The terms “comprising”, “including”, and “having” are intended to be inclusive and should be interpreted as meaning there may be additional elements other than those named.

As various changes could be made in the disclosed constructions, methods, and procedures without departing from the scope of the disclosure, it is intended that all matter contained in the description or shown in the accompanying drawings should be interpreted as illustrative and not in a limiting sense. It should be understood that modifications and variations in the constructions, method, and procedures that fall within the scope of the claims should be interpreted as part of the scope of the invention and as not departing from the scope of the invention.

Unless otherwise expressly stated, it is in no way intended that any method or aspect set forth herein be construed as requiring its steps be performed in a specific order. This construction holds for possible non-express bases for interpretation, including matters of logic with respect to arrangement of steps or operational flow, or plain meaning derived from grammatical organization or punctuation.

Insofar as the written description, the claims, and the accompanying drawings disclose additional subject matter that is not deemed to fall within the scope of the claims, the subject matter is expressly not dedicated to the public and the right to file other applications to claim the subject matter is reserved.

As those skilled in the art could make various changes to the above constructions, products, and methods without departing from the intended scope of the description, all matter in the above description and accompanying drawings should be interpreted as illustrative and not in a limiting sense.

1. A method of constructing a replacement valve for repairing a patient's failing heart valve having an annulus separating an upstream region from a downstream region, said method comprising the steps of:
obtaining a representative inner perimetrical length of the annulus;
fabricating a frame having a central hub and a plurality of flexibly resilient legs extending radially outward from the central hub, each leg of said plurality of legs extending to an anchor axially offset from the central hub by a preselected distance;
fashioning an annular band having an outer circumferential length corresponding to the representative inner perimetrical length of the annulus;
attaching the band to at least a portion of the plurality of legs of the frame adjacent to the respective anchors to limit spacing between the respective anchors of adjacent legs of said plurality of legs;
forming a flexible valve component having a convex face and a concave face opposite said convex face, said convex face having an annular margin and a central region axially offset from the annular margin; and
connecting the central region of the convex face to the central hub of the frame and circumferentially spaced portions of the annular margin to at least one of the band and a portion of the frame adjacent to the anchors;
wherein said valve component is adapted to move when repairing the patient's failing heart valve in response to a difference between fluid pressure in the upstream region and fluid pressure in the downstream region;
wherein the valve component moves relative to the band between an open position in which the valve component permits downstream flow between the band and the annular margin of the valve component and a closed position in which the valve component blocks upstream flow between the band and the annular margin of the valve component; and
wherein the valve component moves to the open position when fluid pressure in the upstream region is greater than fluid pressure in the downstream region to permit downstream flow from the upstream region to the downstream region and the valve component moves to the closed position when fluid pressure in the downstream region is greater than fluid pressure in the upstream region to prevent flow reversal from the downstream region to the upstream region.

2. A method as set forth in claim 1, in which the element of the patient's failing heart valve includes leaflets surrounded by the corresponding annulus in the heart, wherein the step of obtaining the representative inner perimetrical length of the annulus comprises measuring a perimetrical length of the annulus when the leaflets are fully opened and positioned against the annulus.

3. A method as set forth in claim 2, wherein the step of estimating the perimetrical length of the annulus comprises performing a medical procedure before constructing the replacement valve.

4. A method as set forth in claim 3, wherein the medical procedure comprises intracardiac echocardiography.

5. A method as set forth in claim 1, in which the element of the patient's failing heart valve includes leaflets surrounded by the corresponding annulus in the heart, wherein the step of obtaining the representative inner perimetrical length of the annulus comprises estimating a perimetrical length of the annulus when the leaflets are removed from the annulus.

6. A method as set forth in claim 5, wherein the step of estimating the perimetrical length of the annulus comprises performing a medical procedure before constructing the replacement valve.

7. A method as set forth in claim 6, wherein the medical procedure comprises intracardiac echocardiogram.

8. A method as set forth in claim 1, wherein the step of fabricating the frame comprises:
forming a plurality of flexibly resilient U-shaped elements, each U-shaped element of said plurality of U-shaped elements having a central portion separating opposite end portions; and
joining the central portions of said plurality of U-shaped elements to form the central hub, each end portion of the joined U-shaped elements forming one leg of said plurality of legs extending from the central hub.

9. A method as set forth in claim 1, wherein each of said anchors comprises a cleat adapted to retain the replacement valve when the replacement valve is implanted in the patient's heart during a subsequent medical procedure performed after constructing the replacement valve.

10. A method as set forth in claim 1, wherein:
the band comprises an outer strip having an inner face extending around the legs of the frame and an inner strip having an outer face attached to the inner face of the outer strip; and
the outer strip has an outer face having a circumferential length corresponding to the representative inner perimetrical length of the annulus.

11. A method as set forth in claim 1, wherein the flexible valve component is conical.

12. A method as set forth in claim 1, wherein said valve component is substantially free of connections to the frame other than at the central hub of the frame and at said circumferentially spaced portions.

13. A method of performing a medical activity to repair a patient's failing heart having an annulus separating an upstream region from a downstream region, said method comprising the steps of:
performing intracardiac echocardiography to measure a representative inner perimetrical length of the annulus;
constructing a replacement valve using a construction procedure comprising the steps of:
fabricating a frame having a central hub and a plurality of flexibly resilient legs extending radially outward from the central hub, each leg of said plurality of legs extending to an anchor axially offset from the central hub by a preselected distance;
fashioning an annular band having an outer circumferential length corresponding to the measured representative inner perimetrical length of the annulus;
attaching the band to at least a portion of the plurality of legs of the frame adjacent to the respective anchors to limit spacing between the respective anchors of adjacent legs of said plurality of legs;
forming a flexible valve component having a convex face and a concave face opposite said convex face, said convex face having an annular margin and a central region axially offset from the annular margin; and
connecting the central region of the convex face to the central hub of the frame and circumferentially spaced portions of the annular margin to at least one of the band and a portion of the frame adjacent to the anchors;
wherein the valve component is adapted to move when repairing the patient's failing heart valve in response to a difference between fluid pressure in the upstream region and fluid pressure in the downstream region;
wherein the valve component moves relative to the band between an open position in which the valve component permits downstream flow between the band and the annular margin of the valve component and a closed position in which the valve component blocks upstream flow between the band and the annular margin of the valve component; and
wherein the valve component moves to the open position when fluid pressure in the upstream region is greater than fluid pressure in the downstream region to permit downstream flow from the upstream region to the downstream region and the valve component moves to the closed position when fluid pressure in the downstream region is greater than fluid pressure in the upstream region to prevent flow reversal from the downstream region to the upstream region; and
performing heart surgery to implant the constructed replacement valve in the patient's failing heart with the annular band of the replacement valve aligned with the measured annulus separating the upstream region from the downstream region and the flexible valve component oriented so the valve component moves to the open position when fluid pressure in the upstream region is greater than fluid pressure in the downstream region and the valve component moves to the closed position when fluid pressure in the downstream region is greater than fluid pressure in the upstream region.

14. A method as set forth in claim 13, in which the element of the patient's failing heart valve includes leaflets surrounded by the annulus, wherein the step of measuring the representative inner perimetrical length of the annulus comprises measuring a perimetrical length of the annulus when the leaflets are fully opened and positioned against the annulus.

15. A method as set forth in claim 13, in which the element of the patient's failing heart valve includes leaflets surrounded by the annulus, wherein the step of measuring the representative inner perimetrical length of the annulus comprises estimating a perimetrical length of the annulus when the leaflets are removed from the annulus.

16. A method of constructing a replacement valve for repairing a patient's failing heart valve having an annulus separating an upstream region from a downstream region, said annulus having a known representative inner perimetrical length, said method comprising the steps of:
fabricating a frame having a central hub and a plurality of flexibly resilient legs extending radially outward from the central hub, each leg of said plurality of legs extending to an anchor axially offset from the central hub by a preselected distance;
fashioning an annular band having an outer circumferential length corresponding to the representative inner perimetrical length of the annulus;
attaching the band to at least a portion of the plurality of legs of the frame adjacent to the respective anchors to limit spacing between the respective anchors of adjacent legs of said plurality of legs;
forming a flexible valve component having a convex face and a concave face opposite said convex face, said convex face having an annular margin and a central region axially offset from the annular margin; and
connecting the central region of the convex face to the central hub of the frame and circumferentially spaced portions of the annular margin to at least one of the band and a portion of the frame adjacent to the anchors;
wherein said valve component is adapted to move when repairing the patient's failing heart valve in response to a difference between fluid pressure in the upstream region and fluid pressure in the downstream region;
wherein the valve component moves relative to the band between an open position in which the valve component permits downstream flow between the band and the annular margin of the valve component and a closed position in which the valve component blocks upstream flow between the band and the annular margin of the valve component; and
wherein the valve component moves to the open position when fluid pressure in the upstream region is greater than fluid pressure in the downstream region to permit downstream flow from the upstream region to the downstream region and the valve component moves to the closed position when fluid pressure in the downstream region is greater than fluid pressure in the upstream region to prevent flow reversal from the downstream region to the upstream region.

17. A method as set forth in claim 16, wherein the step of fabricating the frame comprises:
forming a plurality of flexibly resilient U-shaped elements, each U-shaped element of said plurality of U-shaped elements having a central portion separating opposite end portions; and
joining the central portions of said plurality of U-shaped elements to form the central hub, each end portion of the joined U-shaped elements forming one leg of said plurality of legs extending from the central hub.

18. A method as set forth in claim 16, wherein each of said anchors comprises a cleat adapted to retain the replacement valve when the replacement valve is implanted in the patient's heart during a subsequent medical procedure performed after constructing the replacement valve.

19. A method as set forth in claim 16, wherein:
the band comprises an outer strip having an inner face extending around the legs of the frame and an inner strip having an outer face attached to the inner face of the outer strip; and
the outer strip has an outer face having a circumferential length corresponding to the representative inner perimetrical length of the annulus.

20. A method as set forth in claim 16, wherein said valve component is substantially free of connections to the frame other than at the central hub of the frame and at said circumferentially spaced portions.
